(19) United States
(12) Patent Application Publication
(10) Pub. No.: US 2010/0048488 A1
(43) Pub. Date: Feb. 25, 2010

(54) IMMUNOMODULATORY PEPTIDES

(75) Inventors: Adam R. Mezo, Needham, MA (US); Kevin A. McDonnell, Waltham, MA (US); Ryan Donnelly, Somerville, MA (US)

Correspondence Address: BIOGEN IDEC / FINNEGAN HENDERSON, LLP, 901 NEW YORK AVENUE, NW, WASHINGTON, DC, 20001-4413, US

(73) Assignee: Syntonix Pharmaceuticals, Inc.

(21) Appl. No.: 12/533,474

(22) Filed: Jul. 31, 2009

Related U.S. Application Data
(60) Provisional application No. 61/085,547, filed on Aug. 1, 2008.

(51) Int. Cl. C07K 7/08; A61K 38/17; G01N 33/53; A61P 37/06; A61P 37/08; A61P 7/06; A61P 17/06; A61P 1/00; A61P 1/04; A61P 9/00; A61P 9/10

(52) U.S. Cl. 514/14; 530/327; 436/501; 435/7.9; 530/413

(57) ABSTRACT

The invention relates to peptides which, in some embodiments, bind to human FcRn and inhibit binding of the Fc portion of an IgG to an FcRn, thereby modulating serum IgG levels. The disclosed compositions and methods may be used in some embodiments, for example, in treating autoimmune diseases and inflammatory disorders. The invention also relates, in further embodiments, to methods of using and methods of making the peptides of the invention.

IgG plays a critical role in mediating protection against pathogens and in mediating allergic and inflammatory responses that hasten recruitment of immune system components to the tissues, mucosae, and dermal surfaces. Junghans, Immunol. Res. 16(1):29 (1997). However, IgG also plays a key role in a variety of autoimmune diseases.

The serum half-life of IgG is longer than the serum half-lives of other plasma proteins. For example, the serum half-life of IgG is 5 to 7 days in mice and 22 to 23 days in humans. Roopenian et al., J. Immunol. 170:3528 (2003); Junghans and Anderson, Proc. Natl. Acad. Sci. USA 93:5512 (1996). That extended serum half-life is at least partly due to the neonatal Fc receptor, FcRn, which binds to the Fc portion of pinocytosed IgG (in both adults and neonates) to protect it from lysosomal degradation. The pinocytosed IgG is then recycled back to the extracellular compartment. See, e.g., Junghans and Anderson, Proc. Natl. Acad. Sci. USA 93:5512 (1996), Roopenian et al., J. Immunol. 170:3528 (2003). Indeed, the serum half-life of IgG is reduced in knockout mouse models that do not express at least part of the genes encoding β₂m and FcRn heavy chain. See WO 02/43658 and Junghans and Anderson, Proc. Natl. Acad. Sci. USA 93:5512 (1996).

When the concentration of IgG reaches a level that exceeds available FcRn, unbound IgG is not protected from degradative mechanisms and consequently has a shorter serum half-life. See, e.g., Brambell et al., Nature 203:1352 (1964). Analogously, IgG serum half-life is reduced when IgG binding to FcRn is inhibited, thereby preventing IgG recycling. Therefore, agents that inhibit or antagonize the binding of IgG to FcRn may be used for regulating, treating or preventing disorders characterized by the presence of inappropriately expressed IgG antibodies (such as, e.g., autoimmune and inflammatory diseases and disorders). For example, antibodies capable of inhibiting the binding of FcRn with IgG have been generated using a FcRn heavy chain knockout mouse line (WO 02/43658). In another example, peptides have been identified that bind to FcRn complexes. Kolonin et al., Proc. Natl. Acad. Sci. USA 99(20):13055-60 (2002); U.S. Pat. No. 6,212,022. The contents of U.S. application Ser. No. 11/676,148, filed Feb. 16, 2007, and U.S. Provisional Application Nos. 60/774,853, filed Feb. 17, 2006, and 60/805,634, filed Jun. 23, 2006, describing further such peptides, their synthesis, and their uses are herein incorporated by reference in their entirety. However, at this time additional agents are needed to regulate, treat, or prevent conditions, diseases, and disorders characterized by immune reactions.

Accordingly, peptides which specifically bind to FcRn and inhibit IgG Fc from binding to FcRn, thereby preventing IgG from recycling by preventing FcRn from functioning in its role of protecting IgG from degradation by the lysosomes are disclosed. In exemplary embodiments, the peptides bind to FcRn and inhibit the IgG1, IgG2, IgG3, or IgG4 subclasses of IgG from binding to FcRn.

In some embodiments, the invention provides pharmaceutical compositions comprising a therapeutically effective amount of one or more peptides of the invention.

In other embodiments, the invention provides methods of regulating a disease state comprising contacting a cell with a therapeutically effective amount of one or more peptides of the invention. Further embodiments include methods of regulating IgG levels in the serum of a subject comprising administering to the subject a therapeutically effective amount of a composition comprising one or more peptides of the invention capable of binding to and inhibiting the FcRn from binding to the Fc portion of an IgG molecule. In certain embodiments, the methods of the invention may be employed to reduce the half-life of soluble IgG in the serum of a subject. In some embodiments, the result of administering a composition of the invention is that the half-life of soluble IgG in the serum of the subject is reduced compared to the half-life of IgG in the serum of the subject prior to administration of the peptide.

In other embodiments, the invention provides methods for inhibiting binding of the Fc portion of a human IgG to FcRn to effect a decrease in the serum concentration of IgG as compared to the serum concentration of IgG before treatment. The method of decreasing serum concentration of IgG comprises administering to the subject a therapeutically effective amount of a composition comprising one or more peptides of the invention that inhibit binding of the Fc portion of an IgG molecule to FcRn. In some embodiments, the decrease in the serum concentration of human IgG is at least 5%, such as a decrease of at least 15%, or a decrease in the serum concentration of human IgG of at least 25%.

Some embodiments of the invention provide methods of treating a subject suffering from a disease characterised by increased or inappropriate expression of IgG, such as, e.g., an an autoimmune disease, an inflammatory disease, or an immune system cancer, comprising administering to the subject a therapeutically effective amount of a composition comprising one or more peptides of the invention capable of preventing the FcRn from binding to the Fc portion of an IgG molecule. In some embodiments, methods of the invention may be used to prevent, treat, or regulate an immune response to a therapeutic protein or a gene therapy vector.

In other embodiments, methods of detecting FcRn are provided, comprising labeling a peptide described herein with at least one detectable label chosen from, e.g., a radioisotope, an enzyme (e.g., an enzyme that catalyzes a reaction producing a detectable, including, e.g., a colored, luminescent, or fluorescent, product), a fluorophore, a chromophore, a chemiluminescent compound, a magnetic particle, a microsphere, a nanosphere, biotin, streptavidin, and digoxin.

Other embodiments of the invention include methods of purifying FcRn, comprising immobilizing a peptide described herein to a solid support, contacting a solution containing FcRn with the immobilized peptide on a solid support; and purifying FcRn by separating the solution from said solid support.

Additional embodiments, objects, and advantages of the invention are set forth in part in the description which follows and in part, will be obvious from the description, or may be learned by practice of the invention. These embodiments, objects, and advantages of the invention may be realized and attained by means of the elements and combinations particularly pointed out in the appended claims.

It is to be understood that both the foregoing general description and the following detailed description are only exemplary and explanatory and are not restrictive of the invention as claimed.

### DESCRIPTION OF THE EMBODIMENTS

The invention is based, in part, on the surprising discovery that the addition of a lysine residue to a dimeric anti-FcRn peptide (Peptide No. 283) improves the solubility of the peptide at the physiologically relevant pH 7.4, whereas the addition of an arginine residue to the same dimeric anti-FcRn peptide does not improve the solubility of the peptide at pH 7.4.

It is well known in the art that the addition of a positive charge to a molecule may improve the molecule's solubility. According to this principle, it is generally understood that the addition of an arginine residue to a peptide will improve solubility. It was surprisingly discovered that the addition of an arginine to Peptide No. 283 (described in U.S. application Ser. No. 11/676,148) did not improve the solubility of the peptide. In contrast, the addition of a lysine residue did improve the solubility of Peptide No. 283. This result is particularly surprising because arginine is believed to be more solubilizing that lysine. See, e.g., page 18 of Kato et al., Biopolymers 85(1):12-18 (2006).

### I. Definitions

The term “amino acid,” as used herein, encompasses encoded and non-encoded amino acids. Standard 1- and 3-letter abbreviations are used herein for the encoded amino acids (alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine).

Non-encoded amino acids include, e.g., α-amino acids, β-amino acids, γ-amino acids, δ-amino acids, and ω-amino acids, and may have R or S chirality at any chiral atom. Non-encoded amino acids include isomers of the encoded amino acids such as, e.g., stereoisomers (including, e.g., D-amino acids and allo-amino acids such as, e.g., allo-threonine and allo-isoleucine) and structural isomers (including, e.g., β-alanine) of the encoded amino acids. Lower case single-letter codes are used herein to indicate stereoisomers of the encoded amino acids having D-chirality (e.g., a=D-alanine, y=D-tyrosine). Non-encoded amino acids also include N-methylated amino acids. Conventional 3-letter abbreviations are used herein for certain common non-encoded amino acids (e.g., Aib=aminoisobutyric acid, Apa=5-aminopentanoic acid, Dab=1,3-diaminobutyric acid, Dap=1,2-diaminopropionic acid, Orn=ornithine, Pen=penicillamine, Sar=sarcosine). In general, where no specific configuration is indicated for an α-amino acid, one skilled in the art would understand that amino acid to be an L-amino acid. However, in particular embodiments, non-encoded amino acids may also be in the form of racemic, non-racemic, and diastereomeric mixtures.

Non-encoded amino acids are well known in the peptide art and include, e.g., N-acetylserine, allo-isoleucine, allo-threonine, β-alanine (3-aminopropionic acid), α-aminoadipic acid, 2-aminobutanoic acid, 4-aminobutanoic acid, 3-amino-1-carboxymethylvalerolactam, 1-aminocyclopentanecarboxylic acid, 6-aminohexanoic acid, 2-aminoheptanedioic acid, 7-aminoheptanoic acid, 2-aminoisobutyric acid, aminomethylpyrrole carboxylic acid, 8-amino-3,6-dioxa-octanoic acid, aminopiperidinecarboxylic acid, aminoserine, aminotetrahydropyran-4-carboxylic acid, azetidine carboxylic acid, benzothiazolylalanine, butylglycine, camitine, 4-chlorophenylalanine, citrulline, cyclohexylalanine, cyclohexylstatine, 2,4-diaminobutanoic acid, 2,3-diaminopropionic acid, dihydroxyphenylalanine, dimethylthiazolidine carboxylic acid, 4-guanyl-phenylalanine, homoarginine, homocitrulline, homocysteine, homophenylalanine, homoproline, homoserine, 4-hydrazinobenzoic acid, 4-hydroxyproline, isonipecotic acid, methanoproline, norleucine, norvaline, ornithine, p-aminobenzoic acid, penicillamine, phenylglycine, O-phosphoserine, piperidinylalanine, piperidinylglycine, pyrrolidinylalanine, sarcosine, statine, tetrahydropyranglycine, thienylalanine, ε-N,N,N-trimethyllysine.

### II. Peptides

The peptides of the invention may be monomeric or multimeric, wherein each of the individual peptide monomers from which the multimer is composed are the same. For example, peptide dimers may be synthesized by reacting individual peptide monomers, while on resin, with a di- or bivalent linker. In other embodiments, peptide multimers may be synthesized by incorporating branched linker groups prior to the synthesis of the peptide sequence as in, e.g., Posnett et al., J. Biol. Chem. 263:1719 (1988).

In an exemplary embodiment, a peptide of the invention has the sequence

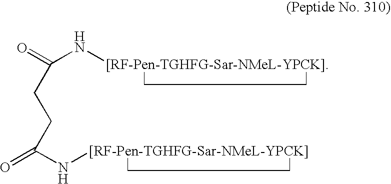

In the sequence above, Pen=penicillamine; Sar=sarcosine; NMeL=N-methylleucine; and horizontal brackets placed below the peptide sequence indicate the presence of a bridge. The small vertical brackets identify the individual peptide monomers of the invention that form the dimeric peptide. Optionally, each monomer of the peptides may be modified to include one or more additional lysine residues at its carboxy terminus.

In some embodiments, the disclosure provides peptides derivatized with a hydrophilic polymer as described in U.S. Provisional Application No. 60/954,968 (now published as WO 2009/020867), the contents of which are herein incorporated by reference. For example, any of the peptides disclosed in the Examples may be derivatized with a hydrophilic polymer or may be modified (e.g., as described below) so that they can be derivatized with a hydrophilic polymer. The term “derivatized,” as used in connection with the peptides of the invention, refers to amino acids or peptides, or analogs of amino acids or peptides, comprising a hydrophilic polymer.

The hydrophilic polymer may be chosen from, e.g., polyethylene glycol including, e.g., monoalkyl-polyethylene glycol; polypropylene glycol; polysaccharides such as, e.g., dextran and cellulose; methylcellulose; hydroxycellulose; hydroxymethylcellulose; hydroxypropylcellulose; hydroxypropylmethyl cellulose; hydroxyalkyl starch including, e.g., hydroxyethyl starch; polyvinyl alcohol; poly(N-vinyl pyrrolidone); and poloxamers. In other embodiments, the hydrophilic polymer may be chosen from, e.g., polyethylene glycol copolymers such as, e.g., polyethylene glycol-polypropylene glycol copolymers and polyethylene glycol-poly(N-vinyl pyrrolidone) copolymers. In some embodiments, the hydrophilic polymer is a non-peptide polymer. In some embodiments, the hydrophilic polymer is readily hydrated. In some embodiments, the hydrophilic polymer has a large hydrodynamic radius when hydrated. In illustrative embodiments, the hydrophilic polymer is polyethylene glycol.

In some embodiments, a peptide of the invention (a monomer or multimer of

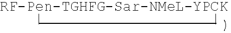

may contain one molecule of hydrophilic polymer per peptide monomer. In other embodiments, a peptide of the invention may contain multiple molecules of hydrophilic polymer per peptide monomer. For example, the anti-FcRn peptides disclosed herein may have 1, 2, 3, 4, 5, 6, 7, 8, or 1-4, 1-8, 2-3, 2-4, 2-6, 3-6, or 2-6 molecules of hydrophilic polymer per peptide monomer.

In some embodiments, the hydrophilic polymer may be linear. In other embodiments, the hydrophilic polymer may be branched. A branched hydrophilic polymer may have, e.g., 2, 3, 4, 5, 6, 7, or 8 branches. In some embodiments, the hydrophilic polymer may have an average molecular weight of, e.g., about 5, 10, 15, 20, 25, 30, 35, 40, 45, 50, 55, or 60 kDa, or may have an average molecular weight ranging from, e.g., about 10-60, 10-40, 10-30, 20-30, 20-40, 20-50, 30-60, 15-25, 25-35, 35-45, or 45-55 kDa.

The peptides may contain a bridge. The bridge may include, or may result from the formation of, one or more functional groups such as, e.g., a disulfide, an ether, a thioether, an alkene, or an amide, in which case the bridge may be referred to as, e.g., a disulfide, ether, thioether, alkene, or amide bridge.

Any suitable linker known to one of skill in the art may be used. In general, linkers that do not interfere with binding to FcRn are chosen. For example, the linker may be one of the linkers disclosed in, e.g., the Examples; U.S. Pat. Nos. 4,671,958; 4,867,973; 5,691,154; 5,846,728; 6,472,506; 6,541,669; 7,141,676; 7,176,185; and 7,232,805 and in U.S. Patent Application Pub. No. 2006/0228348.

In general, the linker may be of a suitable length such that it avoids steric hindrance between the peptide monomers of the multimer, and does not interfere with the binding of the peptide monomers to FcRn. In some embodiments, the linker is a covalent bond. In other embodiments, the linker may comprise 1-100, 1-60, 5-60, 5-40, 2-50, 2-20, 5-10, or 5-20 linear atoms, where the linker is attached to a peptide monomer by means of, e.g., an ester, amide, hydrazone, oxime, semicarbazone, ether, thioether, phosphorothioate, phosphonate, thioester, and/or disulfide linkage. The remaining linear atoms in the linker are preferably selected from the group consisting of carbon, oxygen, nitrogen and sulfur, any of which atoms optionally may be included in a carbocyclic, heterocyclic, aryl, or heteroaryl ring. The linear carbon atoms in the linker optionally can be substituted with a substituent selected from the group consisting of halo, hydroxy, nitro, haloalkyl, alkyl, alkaryl, aryl, aralkyl, alkoxy, aryloxy, amino, acylamino, alkylcarbamoyl, arylcarbamoyl, aminoalkyl, alkoxycarbonyl, carboxy, hydroxyalkyl, alkanesulfonyl, arenesulfonyl, alkanesulfonamido, arenesulfonamido, aralkylsulfonamido, alkylcarbonyl, acyloxy, cyano, and ureido. A linear nitrogen atom in the linker optionally can be substituted with acyl, sulfonyl, alkyl, alkaryl, aryl, aralkyl, alkoxycarbonyl. A linear sulfur atom in the linker optionally can be oxidized. In certain embodiments, the linker may be cleavable, as disclosed in, e.g., U.S. Patent Application Pub. No. 2006/0228348 and U.S. Pat. Nos. 4,867,973; 7,176,185; 7,232,805.

In some embodiments, peptides of the invention are provided as conjugates, including, e.g., covalent and non-covalent conjugates, comprising a peptide and a second molecule, which may be, e.g., a protein, a peptide, a small molecule, a polymer, or a nucleic acid. In some embodiments, the second molecule may confer a desired property to a peptide described herein, such as, e.g., extended half-life, stability, and/or enhanced transport. In some embodiments, the second molecule may enhance the efficacy of a peptide of the invention, as measured by, e.g., the IgG competition ELISA as shown in Example 4. In some embodiments, the second molecule may enhance the efficacy of a peptide of the invention, as measured by, e.g., overall reduction in serum IgG levels in cynomolgus monkeys or by comparison of the frequency of administration of conjugated peptide needed to obtain a particular therapeutic effect, as compared to the unconjugated peptide. In further embodiments, for example, the second molecule may result in targeting of the peptide to a particular cell, tissue, and/or organ.

In some embodiments, the conjugates may have an increased ability to block the IgG-FcRn. In other embodiments, the conjugates may protect the peptide from degradation and thus enhance the in vivo efficacy of the peptide. In some embodiments, the conjugates may have increased circulation half-lives. In further embodiments, such conjugates may be more efficient in binding and neutralizing other molecules than a peptide of the invention. In other embodiments, conjugates may facilitate purification.

In some embodiments, the second molecule of a conjugated peptide of the invention may be an Fc domain of IgG or a fragment thereof. The IgG may be, e.g., human IgG, such as, e.g., human IgG1, IgG2, or IgG4. In some embodiments, the IgG is an altered or mutated IgG, such as, e.g., a Pro331Ser Fcγ₂ variant, Leu235Ala Fc_{γ4} variant, Leu234Val Fc_{γ1} variant, Leu235Ala Fc_{γ1} variant, or Pro331Ser Fc_{γ1} variant. In some embodiments, the second molecule may be an IgG fragment that comprises, e.g., hinge, CH2, and/or CH3 domains.

In some embodiments, the second molecule of a conjugated peptide of the invention may be albumin, an albumin fragment, or an albumin-binding molecule (such as, e.g., peptides, proteins, and molecules including, e.g., long alkyl chains, that bind non-covalently to albumin). Such conjugates may have longer in vivo half-lives and may thus require a lower peptide doses to achieve the desired therapeutic effect. See, e.g., Chuang et al., Pharm. Res. 19:569 (2002); U.S. Pat. No. 6,685,179.

In some embodiments, the peptides may comprise further modifications, such as, e.g., glycosylation, acetylation, phosphorylation, or lipidation.

Exemplary embodiments of the invention include a monomer, dimer, trimer, or other multimer of the sequence:

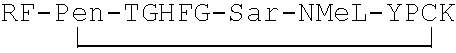

wherein each monomeric peptide is modified to contain one or more additional lysine residues a the carboxy terminus of the sequence and wherein the monomeric or multimeric peptide is modified to contain one or more hydrophilic polymers. In other exemplary embodiments, the one or more hydrophilic polymers are polyethylene glycol. In still other embodiments, the one or more polyethylene glycols are attached to the peptide via a linker. In other exemplary embodiments, the peptide having the following sequence

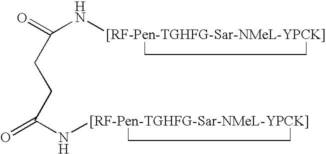

is modified to contain one or more hydrophilic polymers. In certain embodiments, these one or more hydrophilic polymers are polyethylene glycol. In certain embodiments, the sequence is modified to contain one or more additional lysine molecules at the carboxy terminus of each monomer.

The peptides, in certain embodiments, have some affinity for FcRn. For example, in some embodiments, the K_{D} for the peptide-FcRn interaction may range from 50 fM to 1 mM. In other embodiments, the K_{D} may range from 50 fM to 100 μM, 50 fM to 1 nM, or 1 pM to 1 nM.

In some embodiments, the peptides inhibit the Fc portion of IgG from binding to FcRn. For example, in certain embodiments, the peptides can inhibit the Fc portion of IgG from binding to FcRn with an IC₅₀ of, e.g., 50 fM to 100 μM, 50 fM to 1 μM, 1 pM to 100 nM, or 10 pM to 10 nM.

a. Synthesis of Peptides

Peptides of the invention may be synthesized following the procedures set forth in the Examples or by other known synthetic methods, such as, e.g., solid phase peptide synthesis. See, e.g., Abelson et al., eds., Methods in Enzymology, Volume 289: Solid-Phase Peptide Synthesis (1997); Chan and White, eds., Fmoc Solid Phase Peptide Synthesis: A Practical Approach Oxford, University Press Inc., New York (2000); Benoiton, Chemistry of Peptide Synthesis, CRC (2005); Bodanszky, Principles of Peptide Synthesis, 2nd ed., Springer-Verlag, New York (1993); Stewart and Young, Solid Phase Peptide Synthesis, 2nd ed., Pierce Chemical Co., Rockford, Ill. (1984).

Alternatively, peptides of the invention may be synthesized using a combination of synthetic and recombinant methods.

Pegylation may be performed according to any of the pegylation reactions known in the art. Methods for preparing a pegylated protein product will generally include (a) reacting a polypeptide with a PEG containing a first reactive group (such as, e.g., an active ester, aldehyde, amine, aminooxy, hydrazine, hydrazide, othiol, maleimide, and α-haloacyl, such as, e.g., iodoacetyl) under conditions whereby the peptide of the invention, which typically contains at least one second reactive group, becomes attached to one or more PEG groups; and (b) obtaining the reaction product(s). Reaction conditions may be selected from any of those known in the pegylation art or those subsequently developed. In general, reaction conditions (including, e.g., temperature, solvent, and pH) that will not degrade the anti-FcRn peptides of the invention are chosen.

In embodiments wherein a peptide to be pegylated contains more than one second reactive group that may be pegylated, some or all of those groups may be pegylated by using an appropriate PEG stoichiometry during the pegylation reaction. In the illustrative example of a peptide dimer containing two C-terminal amines, both amines may be pegylated, or only one amine may be pegylated, depending upon the PEG stoichiometry used.

Conjugates of the peptides of the invention with proteins, peptides, small molecules, polymers, or nucleic acids may be prepared according to any of the conjugation chemistries known in the art or described herein. For example, in some embodiments, peptides may be capped by a hydrophobic aromatic capping reagents for non-covalent binding to albumin as in, e.g., Zobel et al., Bioorg. Med. Chem. Lett. 13:1513 (2003). In other embodiments, peptides modified with thiol-reactive groups can be used for covalent conjugation to free cysteine residues as in, e.g., Kim et al., Diabetes 52:751 (2003). In further embodiments, a peptide of the invention containing an aldehyde may be reacted with a second molecule by reductive alkylation reaction as in, e.g., Kinstler, Adv. Drug Del. Rev. 54:477 (2002). Alternatively, where the second molecule is a protein or a peptide having an N-terminal cysteine, a peptide thioester may be reacted with the second molecule to form a covalent conjugate as described in, e.g., Dawson and Kent, Annu. Rev. Biochem. 69:923 (2000). Peptide-protein and peptide-peptide conjugates may also, in certain embodiments where all amino acids are encoded amino acids, be prepared by expression in an appropriate host cell.

III. Methods for Assaying Peptides that Bind to FcRn and Block the IgG:FcRn Interaction

A number of methods may be used to assess the ability of a peptide or peptidomimetic to bind FcRn and block the FcRn:IgG interaction. For example, surface plasmon resonance (SPR) is a method well known in the art to evaluate binding events (Biacore AB, Uppsala, Sweden). Using this method, one of the binding partners (FcRn or IgG) is immobilized on the SPR sensor chip and while the other binding partner is passed over the chip, which is monitored for a resulting signal. In the same experiment, the peptide to be evaluated as a competitor of the interaction between IgG and FcRn is passed over the chip. Any decrease in signal may be interpreted as a measure of the peptide's ability to block the interaction between FcRn and IgG.

Other methods for assaying for possible peptide inhibitors of the IgG:FcRn interaction are also well known in the art. One such method is an IgG competition assay in a 96-well plate format. In this example assay, soluble human FcRn on a 96-well plate is exposed to IgG and a test peptide. Residual bound IgG, as detected by an anti-IgG antibody and standard ELISA visualization reagents, provide a measure of the peptide's ability to block the FcRn-IgG interaction.

The ability of a peptide to block IgG-FcRn binding may also be carried out on cells transfected with DNA encoding a human FcRn to develop a cell line capable of expressing human FcRn on its cell surface. A binding competition assay may be applied where peptide inhibitors of IgG-FcRn binding compete with a fluorescently labeled IgG molecule. The level of residual IgG bound to the cells may be measured using, e.g., a standard fluorescent activated cell sorter (FACS).

### IV. Pharmaceutical Uses of Immunomodulatory Peptides

The peptides of the invention bind FcRn and inhibit the Fc portion of the IgG constant region from binding to FcRn resulting in increased catabolism of IgG in comparison to the catabolism of IgG in the absence of peptides of the invention. Typically, these peptides will be in dimeric form, however other multimer forms of the peptides can be used. In exemplary embodiments, the IgG constant region is from the IgG1, IgG2, IgG3, or IgG4 subclasses.

A. Preparation of Pharmaceutical Compositions

The peptides of the invention may be used in the manufacture of a medicament (pharmaceutical composition) for the treatment of any disease or condition where increased catabolism of IgG may be desired. Accordingly, the invention provides pharmaceutical compositions comprising at least one peptide of the invention. These compositions will typically include a pharmaceutically acceptable carrier or excipient. Examples of suitable pharmaceutical carriers are described in Remington's Pharmaceutical Sciences by E. W. Martin. Examples of excipients can include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition can also contain pH buffering reagents, and wetting or emulsifying agents.

The pharmaceutical compositions of the invention may be formulated for administration to a patient in need thereof by any reasonable route of administration, including e.g., intravenously, subcutaneously, intra-muscularly, orally, sublingually, buccally, sublingually, nasally, rectally, vaginally or by inhalation. In some embodiments the peptides of the may be implanted within or linked to a biopolymer solid support that allows for the slow release of the peptide.

For oral administration, the pharmaceutical composition may take the form of tablets or capsules prepared by conventional means. The composition can also be prepared as a liquid, for example as a syrup or a suspension. The liquid can include suspending agents (e.g., sorbitol syrup, cellulose derivatives or hydrogenated edible fats), emulsifying agents (lecithin or acacia), non-aqueous vehicles (e.g., almond oil, oily esters, ethyl alcohol, or fractionated vegetable oils), and preservatives (e.g., methyl or propyl-p-hydroxybenzoates or sorbic acid). The preparations can also include flavoring, coloring and sweetening agents. Alternatively, the composition can be presented as a dry product for constitution with water or another suitable vehicle.

For buccal and sublingual administration the composition may take the form of tablets or lozenges according to conventional protocols.

For administration by inhalation, the compounds for use according to the present invention are conveniently delivered in the form of an aerosol spray from a pressurized pack or nebulizer (e.g., in PBS), with a suitable propellant, e.g., dichlorodifluoromethane, trichlorofluoromethane, dichlorotetrafluoromethane, carbon dioxide or other suitable gas. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The pharmaceutical composition can be formulated for parenteral administration (including, e.g., intravenous or intramuscular administration) by bolus injection. Formulations for injection can be presented in unit dosage form, e.g., in ampoules or in multidose containers with an added preservative. The compositions can take such forms as suspensions, solutions, or emulsions in oily or aqueous vehicles, and contain formulatory agents such as suspending, stabilizing and/or dispersing agents. Alternatively, the active ingredient can be in powder form for constitution with a suitable vehicle, such as, e.g., pyrogen free water.

The pharmaceutical composition can also be formulated for rectal administration as a suppository or retention enema, e.g., containing conventional suppository bases such as cocoa butter or other glycerides.

B. Exemplary Pharmaceutical Compositions

In some embodiments, the invention relates to a pharmaceutical composition comprising a therapeutically effective amount of a peptide of the invention.

In some embodiments, the invention relates to a composition wherein the therapeutically effective amount of the peptide is capable of decreasing the serum concentration of human IgG as compared to the serum concentration of human IgG before treatment with the peptide. In some embodiments, the decrease in the serum concentration of human IgG is at least 5%, 15%, or 25%.

C. Methods of Treatment

The pharmaceutical compositions of the invention are useful to treat any disease or condition, where increased catabolism of IgG is desirable. Thus, the invention provides methods of treating diseases characterized by inappropriately expressed IgG antibodies or undesired amounts or levels of IgG, comprising administering a therapeutically effective amount of a peptide of the invention to a patient in need thereof. In some embodiments, the invention provides methods for treating a disease by modulating the serum concentration of IgG with the peptides of the invention. The terms “treat,” treatment,” and “treating” refer to (1) a reduction in severity or duration of a disease or condition, (2) the amelioration of one or more symptoms associated with a disease or condition without necessarily curing the disease or condition, or (3) the prevention of a disease or condition.

In certain embodiments, the methods of the invention may be employed to treat, prevent, or regulate autoimmune diseases including, but not limited to alopecia areata, ankylosing spondylitis, antiphospholipid syndrome, autoimmune Addison's disease, autoimmune hemolytic anemia, autoimmune hepatitis, autoimmune lymphoproliferative syndrome, autoimmunethrombocytopenic purpura, Behcet's disease, bullous pemphigoid, cardiomyopathy, celiac sprue-dermatitis herpetiformis, chronic fatigue immune dysfunction syndrome, chronic inflammatory demyelinating polyneuropathy, cicatricial pemphigoid, CREST syndrome, cold agglutinin disease, Crohn's disease, Degos' disease, dermatomyositis, dermatomyositis-juvenile, discoid lupus, essential mixed cryoglobulinemia, fibromyalgia-fibromyositis, Graves' disease, Guillain-Barré syndrome, Hashimoto's thyroiditis, idiopathic pulmonary fibrosis, idiopathic thrombocytopenia purpura, IgA nephropathy, insulin dependent diabetes, juvenile arthritis, lichen planus, lupus, Ménière's disease, mixed connective tissue disease, multiple sclerosis, myasthenia gravis, pemphigus (including, e.g., pemphigus vulgaris), pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis/giant cell arteritis, transplant rejection, ulcerative colitis, uveitis, vasculitis, vitiligo, and Wegener's granulomatosis.

In some embodiments, the autoimmune disease is chosen from bullous pemphigoid, idiopathic thrombocytopenia purpura, myasthenia gravis, pemphigus (including, e.g., pemphigus vulgaris), and transplant rejection.

In certain embodiments, compositions comprising the peptides of the invention may be used in combination with steroids for immunosuppression.

The peptides of the invention may be used to treat inflammatory disorders including, but not limited to, asthma, ulcerative colitis and inflammatory bowel syndrome allergy, including allergic rhinitis/sinusitis, skin allergies (including, e.g., urticaria (i.e., hives), angioedema, atopic dermatitis), food allergies, drug allergies, insect allergies, mastocytosis, arthritis, including osteoarthritis, rheumatoid arthritis, and spondyloarthropathies. In some embodiments, the invention provides methods of treating cardiovascular disease with an inflammation-based etiology (e.g., arterial sclerosis), transplant rejection, and/or graft versus host disease (GVHD).

Other embodiments of the invention include methods of treating cancer by administering a peptide of the invention. The methods of the invention may be employed to treat or help regulate cancers involving overproduction of IgG, such as plasma cell cancers, including multiple myeloma.

Frequently, in diseases or conditions requiring administration of a therapeutic protein, the subject will develop antibodies against the therapeutic protein, which, in turn, prevent the therapeutic protein from be available for its intended therapeutic purpose. Accordingly, the peptides of the invention can be used in combination with the therapeutic protein to enhance the benefit of the therapeutic protein by reducing the levels of IgG; wherein, IgG antibodies are responsible for the decreased bioavailability of a therapeutic protein. Accordingly, some embodiments of the invention provide methods of regulating, treating, or preventing a condition, disease, or disorder resulting from an immune response to a clotting factor comprising contacting a cell with a therapeutically effective amount of any of the peptides disclosed herein, wherein the clotting Factor is chosen from fibrinogen, prothrombin, Factor V, Factor VII, Factor VIII, Factor IX, Factor X, Factor XI, Factor XII, Factor XIII, or von Willebrand's Factor. This method may be used to regulate or treat, or prevent an immune response to a clotting factor in a patient suffering, e.g., from hemophilia A or hemophilia B. In some embodiments, peptides of the present invention block Factor VIII inhibitors. In other embodiments, the method may be used to regulate or treat, or prevent an immune response to, e.g., therapeutic erythropoietin in a patient suffering from pure red cell aplasia. The invention further provides methods of regulating, treating, or preventing an immune reaction to a lysosomal hydrolase, the absence of which results in a lysosomal storage disorder, such as, e.g., α-galactosidase A, acid ceramidase, acid α-L-fucosidase, acid β-glucosidase (glucocerebrosidase) acid β-galactosidase, iduronate-2-sulfatase, α-L-iduronidase, galactocerebrosidase, Acid α-mannosidase, acid β-mannosidase, arylsulfatase B, arylsulfatase A, N-acetylgalactosamine-6-sulfate sulfatase, acid β-galactosidase, acid sphingomyelinase, acid α-glucosidase, β-hexosaminidase B, heparan N-sulfatase, α-N-acetylglucosaminidase, acetyl-CoA:α-glucosaminide, N-acetyltransferase, N-acetylglucosamine-6-sulfate sulfatase, α-N-acetylgalactosaminidase, sialidase, β-glucuronidase, and β-hexosaminidase A.

In other embodiments, the methods of the invention may be employed to treat, prevent, or regulate an immune reaction to a gene therapy vector. Obstacles to the successful implementation of gene therapy for the treatment of a disease or condition also include the development of antibodies specific to the therapeutic protein encoded by the transgene as well as possibly to the vector used to deliver the transgene. Accordingly, in some embodiments, the peptides described herein can be administered in combination with gene therapy to enhance the benefit of the encoded therapeutic protein by reducing the levels of IgG. These methods are particularly useful in situations where IgG antibodies are responsible for the decreased bioavailability of a gene therapy vector or the encoded therapeutic protein. The gene therapy vector may be, e.g., a viral vector such as adenovirus and adeno associated virus. Diseases that can be treated using gene therapy include, but are not limited to, cystic fibrosis, hemophilia, PRCA, muscular dystrophy, or lysosomal storage diseases, such as, e.g., Gaucher's disease and Fabry's disease.

In the methods of the invention, the compositions described herein can be administered via any suitable route, such as, e.g., intravenously, subcutaneously, intra-muscularly, orally, sublingually, buccally, sublingually, nasally, rectally, vaginally or by inhalation. In general, the appropriate dose of a composition described herein will vary depending on the disease or condition to be treated, the severity of the disease or conditions, the subject, including the gender, age, and weight of the subject, the desired outcome, and the particular route of administration used. For example, dosages can range from 0.1 to 100,000 μg/kg body weight. In some embodiments, the dosing range may be 1-10,000 μg/kg. In other embodiments, the dosing range may be 10-1,000 μg/kg. In yet further embodiments, the dosing range is 100-500 μg/kg.

The compositions of the invention may be administered continuously or at specific timed intervals. In vitro assays may be employed to determine optimal dose ranges and/or schedules for administration. Other effective dosages can be readily determined by one of ordinary skill in the art through routine trials establishing dose response curves, for example, the amount of the peptides of the invention necessary to increase or decrease the level of IgG can be calculated from in vivo experimentation. Those of skill will readily appreciate that dose levels can vary as a function of the specific compound, the severity of the symptoms, and the susceptibility of the subject to side effects, and preferred dosages for a given compound are readily determinable by those of skill in the art by a variety of means. For example, those skilled in the art can calculate an appropriate dose using readily available information with respect to the amount necessary to have the desired effect, depending upon the particular agent used.

D. Exemplary Method of Treatment Embodiments

In some embodiments, the invention relates to a method of treating a disease characterized by inappropriately expressed IgG antibodies or excess IgG, comprising administering a pharmaceutical composition of the invention to a patient in need thereof. In exemplary embodiments, the disease is an immune reaction to a therapeutic protein chosen from erythropoietin, a lysosomal hydrolase, the absence of which results in a lysosomal storage disorder, and a clotting factor.

In some embodiments, the lysosomal hydrolase is chosen from the group consisting of α-galactosidase A, acid ceramidase, acid α-L-fucosidase, acid β-glucosidase (glucocerebrosidase), acid β-galactosidase, iduronate-2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, acid sphingomyelinase, acid α-glucosidase, β-hexosaminidase B, heparan N-sulfatase, α-N-acetylglucosaminidase, acetyl-CoA:α-glucosaminide, N-acetyltransferase, N-acetylglucosamine-6-sulfate sulfatase, α-N-acetylgalactosaminidase, sialidase, β-glucuronidase, and β-hexosaminidase A.

In other embodiments, the clotting factor is selected from fibrinogen, prothrombin, Factor V, Factor VII, Factor VIII, Factor IX, Factor X, Factor XI, Factor XII, Factor XIII, and von Willebrand's Factor.

In some embodiments, the IgG is specific for a gene therapy vector.

In some embodiments, the disease is chosen from inflammatory diseases, autoimmune diseases, and cancer.

In some embodiments, the autoimmune disease is chosen from alopecia areata, ankylosing spondylitis, antiphospholipid syndrome, autoimmune Addison's disease, autoimmune hemolytic anemia, autoimmune hepatitis, autoimmune lymphoproliferative syndrome, autoimmune thrombocytopenic purpura, Behcet's disease, bullous pemphigoid, cardiomyopathy, celiac sprue-dermatitis herpetiformis, chronic fatigue immune dysfunction syndrome, chronic inflammatory demyelinating polyneuropathy, cicatricial pemphigoid, CREST syndrome, cold agglutinin disease, Crohn's disease, Degos' disease, dermatomyositis, dermatomyositis-juvenile, discoid lupus, essential mixed cryoglobulinemia, fibromyalgia-fibromyositis, Graves' disease, Guillain-Barré syndrome, Hashimoto's thyroiditis, idiopathic pulmonary fibrosis, idiopathic thrombocytopenia purpura, IgA nephropathy, insulin dependent diabetes, juvenile arthritis, lichen planus, lupus, Ménière's disease, mixed connective tissue disease, multiple sclerosis, myasthenia gravis, pemphigus, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis/giant cell arteritis, transplant rejection, ulcerative colitis, uveitis, vasculitis, vitiligo, and Wegener's granulomatosis.

In some embodiments, the autoimmune disease is chosen from bullous pemphigoid, idiopathic thrombocytopenia purpura, myasthenia gravis, pemphigus, and transplant rejection. In some embodiments, the pemphigus is pemphigus vulgaris.

In some embodiments, the inflammatory disease is chosen from asthma, ulcerative colitis and inflammatory bowel syndrome allergy, including allergic rhinitis/sinusitis, skin allergies, food allergies, drug allergies, insect allergies, mastocytosis, arthritis, including osteoarthritis, rheumatoid arthritis, and spondyloarthropathies. In some embodiments, the skin allergy is chosen from urticaria, angioedema, and atopic dermatitis.

### V. In Vivo Imaging and Detection of FcRn

The peptides of the invention may be used in assays to detect FcRn. In some embodiments, the assay is a binding assay that detects binding of a peptide of the invention with FcRn. In some embodiments, FcRn may be immobilized, and one or more peptides described herein may passed over the immobilized FcRn. In alternative embodiments, one or more peptides may be immobilized, and FcRn may be passed over the immobilized peptide(s). Either FcRn or the peptides of the invention may be detectably labeled. Suitable labels include radioisotopes, including, but not limited to ⁶⁴Cu, ⁶⁷Cu, ⁹⁰Y, ¹¹¹In, ¹²⁴I, ¹²⁵I ¹³¹I, ¹³⁷Cs, ¹⁸⁶Re, ²¹¹At, ²¹²Bi, ²¹³Bi, ²²³Ra, ²⁴¹Am, ²⁴⁴Cm and ^{99m}Tc-MDP; enzymes having detectable products (for example, luciferase, peroxidase, alkaline phosphatase, β-galactosidase, and the like); fluorophores (including, e.g., fluorescein (which may be attached as, e.g., fluorescein isothiocyanate), rhodamine, phycoerythrin, phycocyanin, allophycocyanin, o-phthaldehyde and fluorescamine); fluorescence emitting metals, for example, ¹⁵²Eu, or others of the lanthanide series, attached to the peptides of the invention through metal chelating groups such as EDTA; chemiluminescent compounds, for example, luminol, isoluminol, theromatic acridinium ester, acridinium salts, imidazole, and oxalate esteror; and bioluminescent compounds, for example, luciferin, or aequorin (green fluorescent protein), specific binding molecules, for example, magnetic particles, microspheres, nanospheres, luminescent quantum dot nanocrystals, and the like.

Alternatively, specific-binding pairs may be used in assays to detect FcRn, involving, for example, a second stage antibody or reagent that is detectably labeled and that can amplify the signal. For example, the peptides of the invention can be conjugated to biotin, and horseradish peroxidase-conjugated streptavidin added as a second stage reagent. Digoxin and antidigoxin provide another suitable binding pair. In other embodiments, a second stage antibody can be conjugated to an enzyme such as peroxidase in combination with a substrate that undergoes a color change in the presence of the peroxidase. The absence or presence of binding between peptides of the invention and FcRn can be determined by various methods, including flow cytometry of dissociated cells, microscopy, radiography, fluorimetry, chromogenic detection, phosphor imaging, detection of chemiluminescence on film and scintillation counting. Such reagents and their methods of use are well known in the art.

For in vivo diagnostic applications, specific tissues or even specific cellular disorders that may be characterized, at least in part, by expression of FcRn, may be imaged by administration of a sufficient amount of a labeled peptide of the invention.

A wide variety of metal ions suitable for in vivo tissue imaging have been tested and utilized clinically. For imaging with radioisotopes, the following characteristics are generally desirable: (a) low radiation dose to the patient; (b) high photon yield which permits a nuclear medicine procedure to be performed in a short time period; (c) ability to be produced in sufficient quantities; (d) acceptable cost; (e) simple preparation for administration; and (f) no requirement that the patient be sequestered subsequently. These characteristics generally translate into the following: (a) the radiation exposure to the most critical organ is less than 5 rad; (b) a single image can be obtained within several hours after infusion; (c) the radioisotope does not decay by emission of a particle; (d) the isotope can be readily detected; and (e) the half-life is less than four days (Lamb and Kramer, “Commercial Production of Radioisotopes for Nuclear Medicine,” In Radiotracers For Medical Applications, Vol. 1, Rayudu (Ed.), CRC Press, Inc., Boca Raton, pp. 17-62). In some embodiments, the metal is technetium-99m (^{99m}Tc).

Accordingly, the invention provides a method of obtaining an image of an internal region of a subject which comprises administering to a subject an effective amount of a composition comprising at least one of the peptides of the invention containing a metal in which the metal is radioactive, and recording the scintigraphic image obtained from the decay of the radioactive metal. Likewise, the invention provides methods for enhancing an magnetic resonance (MR) image of an internal region of a subject which comprises administering to a subject an effective amount of a composition comprising at least one of the peptides of the invention containing a metal in which the metal is paramagnetic, and recording the MR image of an internal region of the subject.

In some embodiments, other methods provided herein include a method of enhancing a sonographic image of an internal region of a subject comprising administering to a subject an effective amount of a composition comprising at least one of the peptides of the invention containing a metal and recording the sonographic image of an internal region of the subject. In general, the metal may be any non-toxic heavy metal ion. In certain embodiments, a method of enhancing an X-ray image of an internal region of a subject is also provided which comprises administering to a subject a peptide composition containing a metal, and recording the X-ray image of an internal region of the subject. In general, a radioactive, non-toxic heavy metal ion may be used.

Peptides of the invention may be linked to chelators such as those described in, e.g., U.S. Pat. No. 5,326,856. The peptide-chelator complex may then be radiolabeled to provide an imaging agent for diagnosis or treatment of diseases or conditions involving the regulation of IgG levels. The peptides of the invention may also be used in the methods that are disclosed in U.S. Pat. No. 5,449,761 for creating a radiolabeled peptide for use in imaging or radiotherapy.

A. Exemplary Methods of Detecting FcRn

The invention relates to a method of detecting FcRn, comprising: labeling a peptide of the invention with a detectable label chosen from radioisotopes, enzymes having detectable products, fluorophores, chemiluminescent compounds, magnetic particles, microspheres, nanospheres, biotin, streptavidin, and digoxin. In some embodiments, the peptide or conjugate labeled with a detectable label is included in a diagnostic kit.

### VI. Purification of FcRn

The peptides of the invention may also be used to purify FcRn. In some embodiments, the peptide is covalently attached to an appropriate chromatographic matrix to form an efficient FcRn separation media. A solution containing FcRn is then passed over the chromatographic matrix resulting in the non-covalent binding of FcRn to the immobilized binding partner. Solutions containing FcRn may be from biological samples such as a bodily fluid, a tissue or cell sample, cell culture supernatant. The FcRn is purified by washing the immobilized peptide:FcRn complex with a suitable solution to remove impurities and then releasing the FcRn from the chromatographic matrix with a suitable elution solution.

Peptides of the invention can be attached to suitable chromatographic matrices using a number of chemical approaches well known to those skilled in the art. For example, peptides of the invention can be attached to matrices containing suitably reactive groups, such as thiols, amines, carboxylic acids, alcohols, aldehydes, alkyl halides, N-alkylmaleimides, N-hydroxysuccinimidyl esters, epoxides, aminooxys, and hydrazides.

In other embodiments, the peptides of the invention can be modified to contain chemical moieties or peptide sequences that bind non-covalently to an appropriate chromatographic matrix. For example, the peptides could be modified with a biotin moiety and could be non-covalently bound to a chromatographic matrix containing an avidin protein. Alternatively, the modified peptide could be incubated with the FcRn solution and the resulting mixture passed over the appropriate chromatographic matrix to isolate the FcRn:peptide complex.

Examples of similar uses of peptides for affinity purification can be found in Kelley et al, “Development and Validation of an Affinity Chromatography Step Using a Peptide Ligand for cGMP Production of Factor VIII,” In Biotechnology and Bioengineering, Vol. 87, No. 3, Wiley InterScience, 2004, pp. 400-412 and in U.S. Pat. No. 6,197,526.

A. Exemplary Methods of Purifying FcRn

The invention relates to a method of purifying FcRn, comprising:
(a) immobilizing a peptide of the invention to a solid support,
(b) contacting a solution containing FcRn with the immobilized peptide on a solid support; and
(c) purifying FcRn by separating the solution from said solid support.

### Examples

The Examples, which are intended to be purely exemplary of the invention and should therefore not be considered to limit the invention in any way, also describe and detail aspects and embodiments of the invention discussed above. The Examples are not intended to represent that the experiments below are all or the only experiments performed. Efforts have been made to ensure accuracy with respect to numbers used (for example, amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric pressure.

### Example 1

### Expression of Soluble Human FcRn (shFcRn)

Soluble human FcRn cDNA was cloned, expressed and purified as described in the literature using the glutamine synthetase expression system in Chinese hamster ovary (CHO) cells. See, e.g., U.S. Pat. No. 5,623,053. A stop codon was placed after amino acid position 274 in the protein sequence of human FcRn in order to remove the transmembrane region.

### Example 2

### Peptide-IgG Competition ELISA

In order to determine whether the peptides of the invention were also able to block the binding of IgG to FcRn, the following ELISA assay was devised and performed.

A. Biotinylation of shFcRn

A solution of soluble human FcRn (shFcRn) in Tris buffer was dialyzed twice, each time for 3 hours in 2 liters of PBS, pH 8.0. The quantity of recovered shFcRn was determined by measuring the absorbance at 280 nm. The concentration of shFcRn was obtained by multiplying the absorbance reading by the extinction coefficient for shFcRn, which is: ε=85880 M⁻¹cm⁻¹. Biotinylation of shFcRn was accomplished by treating the dialyzed shFcRn with a 2-fold-molar excess of Sulfo-NHS-LC-Biotin (Invitrogen, Carlsbad, Calif.) for 2 hours at 4° C. Afterwards, the shFcRn—Sulfo-NHS-LC-Biotin reaction mixture was dialyzed twice in 2 L of cold PBS, followed by another absorbance reading to determine the concentration of the remaining protein. The biotinylated shFcRn was stored at 4° C. with 0.1 % sodium azide until needed.

B. Peptide-IgG Competition ELISA Assay

96-well ReactiBind Neutravidin-coated plates blocked with BSA (Pierce, Rockford, Ill.) were washed twice with 200 μl/well of Buffer A (Buffer A: PBS pH 7.4 (Gibco, 14040), 0.5% BSA IgG-free, 0.05% Tween-20). The wells were coated with 100 μl/well of 1 μg/ml biotinylated-shFcRn in Buffer A. The plate was sealed and incubated at 37° C. for 2 hours. Afterwards, the plate was washed with 200 μl/well of Buffer B (Buffer B: 100 mM MES pH 6, 150 mM NaCl, 0.5% BSA IgG-free (Jackson ImmunoResearch, West Grove, Pa.), 0.05% Tween-20). Then, 50 μl/well of 6 nM human IgG (Calbiochem, San Diego, Calif.) in Buffer B as well as 50 μl/well of the various peptide competitors (at various concentration) were added, so that the final concentration of IgG in the well was 3 nM. To allow for mixing, the plate was rocked for 2 minutes, sealed and incubated at 37° C. for 2 hours. Following the incubation, the liquid was aspirated from the plate and 100 μl/well of a 1:10 000 dilution of Peroxidase-conjugated goat anti-human IgG F(ab′) fragment-specific F(ab′)₂ fragment (Jackson ImmunoResearch, West Grove, Pa.) in Buffer B was added. The plate was covered, incubated for 30 minutes at room temperature and washed 4 times with 200 μl/well of ice-cold buffer B. SureBlue TMB substrate solution (100 μl/well, KPL, Gaithersburg, Md.) was added and the plate was allowed to incubate at room temperature until color developed, which took 5 to 10 minutes. Once color developed, 100 μl/well of TMB stop solution (KPL, Gaithersburg, Md.) was added and the absorbance was measured at 450 nm. The data was plotted as absorbance vs. peptide concentration to derive the inhibitory concentration 50% (IC₅₀) values.

### Example 3

### Peptide Synthesis

Peptides were synthesized using solid-phase peptide synthesis either manually with a fritted round bottom flask or by using an Advanced Chemtech 396-omega synthesizer (Advanced Chemtech, Louisville, Ky.). Standard Fmoc/tBu protocols were used (W. C. Chan and P. D. White eds., Fmoc Solid Phase Peptide Synthesis: A Practical Approach Oxford University Press Inc. New York (2000)), in combination with a Rink amide resin (Novabiochem, San Diego, Calif.) or PAL-PEG-PS (Applied Biosystems, Foster City, Calif.) to yield C-terminal amides upon cleavage. The coupling reagents were 2-(1H-Benzotriazole-1-yl)-1,1,3,3,-tetramethyluronium hexafluorophosphate (HBTU) and N-hydroxybenzotriazole (HOBt) (Novabiochem, San Diego, Calif.). The base was diisopropylethylamine (DIEA) (Sigma-Aldrich, St. Louis, Mo.), and N,N-dimethylformamide (DMF) was the solvent (EM Science, Kansas City, Mo.). The typical synthesis cycle involved 2×10 minute deprotection steps with 20% piperidine in DMF, 2×30 minute amino acid couplings with HOBt/HBTU and a 10 minute capping step with acetic anhydride/HOBt.

Before the peptides were cleaved from the resin, the N-termini of two peptide monomers were joined with a bi-functional acid linker. For example, Peptide No. 283 was synthesized by reacting the peptide resin containing the peptide sequence analogous to Peptide No. 235 (Arg-Phe-Pen-Thr-Gly-His-Phe-Gly-Sar-NMeLeu-Tyr-Pro-Cys) (with an unprotected N-terminus with 0.5 equivalents of succinic acid (Sigma-Aldrich, St. Louis, Mo.) in the presence of 1 equivalent of PyBOP and 2 equivalents of DIEA. This resulted in adjacent peptides on the resin being covalently attached by amide bonds via their N-termini.

The resulting peptide dimers were cleaved from the resin by treatment for 2 hours with 95% trifluoroacetic acid; 2.5% ethanedithiol; 1.5% triisopropylsilane and 1% water and precipitated with ice-cold ether, centrifuged and triturated three times with ether.

The peptide mixture was concentrated in vacuo and subsequently purified using a Waters Prep600 reversed phase HPLC system (Millford, Mass.) equipped with a 250 mm×21.2 mm Phenomenex (Torrence Calif.) C 18 column. The eluent chosen for the HPLC purification step was a gradient of acetonitrile in water containing 0.1% (w/v) TFA. Appropriate fractions were collected, pooled and lyophilized. Peptide identity and purity was confirmed by reversed phase analytical HPLC in combination with a 250 mm×2 mm column (Phenomenex, Torrence, Calif.) coupled with electrospray mass spectrometry (Mariner ES-MS) (Applied Biosystems, Foster City, Calif.).

The purified reduced peptide was dissolved to ca. 0.1 mg/mL in 10 mM sodium phosphate, pH 7.5 with 20% DMSO and mixed for 3 days at room temperature. This oxidation step permitted the formation of the disulfide bonds within one peptide monomer of the dimer, as opposed to between two monomers of a dimer. The reaction mixture was diluted with water to peptide concentration of 0.05 mg/mL and purified over a C 18 Sep-Pak column (Waters Corp., Milford, Mass.) using an increasing gradient of acetonitrile in water containing 0.1% TFA. The peptide dimer was lyophilized and subjected to analysis by mass spectroscopy (Mariner ES-MS) following liquid chromatography (Applied Biosystems, Foster City, Calif.).

In the case of Peptide No. 283, the disulfide linkage pattern was confirmed by digesting the peptide with trypsin for 30 minutes, then analyzing the resulting peptides by LCMS. Trypsin is known to cleave after arginine and lysine residues, and cleaves Peptide No. 283 at the arginine-phenylalanine bond. The major product of LCMS of Peptide No. 283 is NH₂-[Phe-Pen-Thr-Gly-His-Phe-Gly-Sar-NMeLeu-Tyr-Pro-Cys]-CONH₂(disulfide) (LCMS: M+H=1355.6 Da), which indicates that the disulfide bonds of Peptide No. 283 were formed intramolecularly within each 13 amino acid peptide monomer.

Table 1 provides a listing of dimeric peptides of the invention that contain amide linkers.

Peptide No. 310 was synthesized as described above, except that an additioanl lysine residue was added to the C-terminus. Peptide No. 311 was synthesized as described above, except that an additional arginine residue was added to the C-terminus.

TABLE 1
Dimers With Amide Linkers

| | Sequence^{*} |
|---|---|
| Peptide No. 283 | 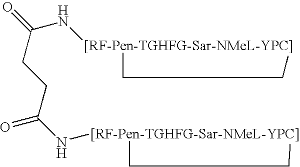 |
| Peptide No. 310 | 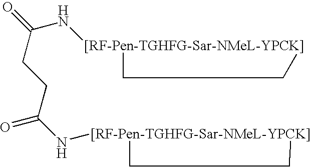 |
| Peptide No. 311 | 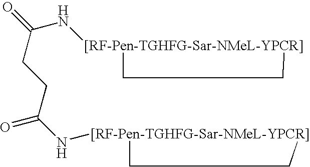 |

^{*}Pen = penicillamine; Sar = sarcosine; NMeL = N-methylleucine; horizontal brackets placed above the peptide sequence indicate the presence of a bridge

### Example 4

### Transgenic Mice

Transgenic mice were obtained from Dr. Roopenian of The Jackson Laboratory in Bar Harbor, Me. The endogenous murine FcRn and β₂m genes were inactivated by insertion of a foreign polynucleotide sequence by homologous recombination and replaced transgenically with the human FcRn and the human β₂m genes (muFcRn (−/−), muβ₂m (−/−), +huFcRn, +huβ₂m). These mice are referred to by the strain name TG32B.

### Example 5

### Effect of Peptide No. 283 and 310 on Human IgG Catabolism in TG32B Mice

Adult TG32B mice were injected intravenously with 500 mg/kg of human IgG (MP Biomedicals, Irvine, Calif.) at t=0 hours (T₀). At 24, 48, 72 and 96 hours, the mice were injected intravenously with 2.5 mg/kg of either Peptide No.283 or Peptide No 310. Control injections were performed at each timepoint using 15 mM sodium acetate, pH 5 and served as the vehicle for Peptide No. 283. The vehicle for Peptide No 310 was PBS+10 mM sodium acetate pH 5. Blood samples were taken prior to injections at all timepoints, 120 hours, and 168 hours. Serum was prepared and stored at −20° C. until an ELISA was performed.

An IgG Fc domain-specific ELISA was used to detect the levels of human IgG in the serum at each time point. Briefly, 30 μl of a 10 μg/ml stock solution of goat anti-human IgG (Pierce, Rockford, Ill.) was diluted with 6 ml of 0.05 M sodium bicarbonate, pH 9.6 (Sigma-Aldrich, St. Louis, Mo.). A 96-well plate was coated with 50 μl/well of this solution and incubated for 1 hour at 37° C. The coating solution was removed and washed once with PBST (phosphate buffered saline with 0.05% Tween-20). Then 200 μl/well of a 2% bovine serum albumin (BSA) stock solution in PBS was added and the plate incubated for 1 hour at 37° C. The wells were washed three times with PBST and a standard curve was generated in triplicate by performing 2.5-fold dilutions starting from 50 ng/ml of hIgG1. Then 100 μl of either the standard or sample solutions was added to the wells and the plate was incubated for 1 hour at 37° C. Three more PBST washes were performed followed by the addition of 100 μl of a 1:10,000 dilution of a goat anti-human IgG[Fc]-HRP conjugate (Pierce, Rockford, Ill.) in PBS containing 2% BSA. The plate was allowed to incubate for 1 hour at 37° C. followed by washes with PBST and the addition of a 100 μl of TMB One-Component substrate (BioFX, Owings Mills, Md.) to each well. Color development was halted after 5 minutes by the addition of 100 μl of 0.25 M sulfuric acid to each well. The UV absorbance for each well was measured at 450 nm and a calibration curve was used to derive a plot of serum IgG concentration vs. time for the experiments. The results are shown in Table 2.

TABLE 2

| | hIgG in serum, % 24 hr levels | | |
|---|---|---|---|
| | | | Peptide No. |
| Time (h) | Vehicle | Peptide No 310 | 283 |
| 24 | 100.0 | 100.0 | 100.0 |
| 30 | 83.1 | 53.1 | 62.5 |
| 48 | 70.1 | 42.9 | 40.7 |
| 72 | 53.7 | 26.1 | 24.2 |
| 96 | 46.8 | 17.7 | 14.4 |
| 120 | 37.7 | 11.6 | 9.8 |
| 168 | 32.4 | 9.3 | 7.1 |

### Example 6

### In Vitro Activity and Solubility of Peptide Nos. 283, 310, and 311

Peptide Nos 283, 310, and 311 were evaluated in the Peptide-IgG competition assay described in Example 3. The solubility of these peptides at the high concentrations of 50 mg/mL and 100 mg/mL was determined in two different sets of conditions as shown in Table 2, whereby the buffer of interest was added to each of the lyophilized peptides and solubility was determined by visual inspection (appearance) of the solution after 30 minutes at room temperature. The results are shown in Table 3.

TABLE 3
In Vitro Potency and Solubility Data

| | | | Solubility at |
|---|---|---|---|
| | | Solubility at 50 mg/mL | 100 mg/mL |
| | | peptide in PBS + 40 mM | peptide in 100 mM |
| | IgG | sodium phosphate, pH | sodium phosphate, pH |
| | Competition | 7.4, after 30 minutes at | 7.4, after 30 minutes at |
| Peptide | ELISA (nM) | room temperature | room temperature |
| 283 | 2.4 | Insoluble | Insoluble |
| 310 | 1.8 ± 1.1 | Soluble | Soluble |
| 311 | 1.5 ± 0.7 | Insoluble | Insoluble |

### Example 7

### Pharmacokinetics of Peptide 283 and Peptide 310 in SD Rats

SD Rats (3 per group) were treated with a single subcutaneous dose of either Peptide 283 or Peptide 310, at a peptide concentration of 30 mg/mL. Blood was collected at 0.5, 2, 4, 6, 24, 48 hours, serum was prepared and the peptide concentrations in serum were determined by LCMS.

TABLE 4

| Timepoint | Peptide 283 | Peptide 310 |
|---|---|---|
| (hr) | ng/mL | ng/mL |
| 0.5 | 23 ± 14 | 137 ± 60 |
| 2.00 | 50 ± 16 | 183 ± 81 |
| 4.00 | 66 ± 42 | 440 ± 156 |
| 6.00 | 68 ± 34 | 557 ± 268 |
| 24.00 | 41 ± 6 | 117 ± 36 |
| 48.00 | 19 ± 4 | 37 ± 15 |

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications and patents cited in this disclosure are incorporated by reference in their entirety. To the extent the material incorporated by reference contradicts or is inconsistent with this specification, the specification will supersede any such material. The citation of any references herein is not an admission that such references are prior art to the present invention.

Unless otherwise indicated, all numbers expressing quantities of ingredients, reaction conditions, and so forth used in the specification, including claims, are to be understood as being modified in all instances by the term “about.” Accordingly, unless otherwise indicated to the contrary, the numerical parameters are approximations and may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the following claims.

1. A peptide comprising the sequence:

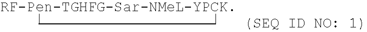

2. The peptide of claim 1 in multimeric form.

3. The peptide of claim 1, wherein the peptide is a dimer.

4. A peptide comprising the sequence:

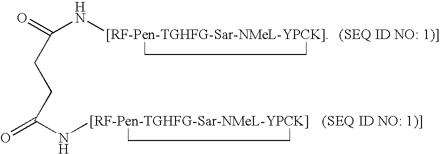

5. The peptide of claim 2 modified to contain a linker.

6. The peptide of claim 2 modified to contain one or more hydrophilic polymers.

7. The peptide of claim 6, wherein the one or more polymers is selected from polyethylene glycol, polypropylene glycol; polysaccharides, hydroxyl alkyl starch; poloxamers, and polyethylene glycol copolymers.

8. The peptide of claim 2 conjugated to a second molecule.

9. The peptide of claim 2 conjugated to an Fc domain of IgG or a fragment thereof.

10. The peptide of claim 2 including one or more additional lysine residues at the carboxy end of each monomer.

11. A pharmaceutical composition comprising a therapeutically effective amount of the peptide of claim 2.

12. A method of treating a disease or disorder characterized by inappropriately expressed IgG antibodies or excess IgG, comprising administering the composition of claim 11 to a patient in need thereof.

13. The method of claim 12, wherein the disease or disorder is selected from an immune reaction to a therapeutic protein, an inflammatory disorder, an autoimmune disease, and cancer.

14. The method of claim 13, wherein the inflammatory disease or disorder is selected from asthma, ulcerative colitis, inflammatory bowel syndrome, allergies, allergic rhinitis/sinusitis, skin allergies, urticaria, angioedema, atopic dermatitis, food allergies, drug allergies, insect allergies, mastocytosis, osteoarthritis, rheumatoid arthritis, spondyloarthropathies, cardiovascular disease with an inflammation-based etiology, arterial sclerosis, transplant rejection, andr graft versus host disease.

15. The method of claim 13, wherein the autoimmune disease or disorder is selected from alopecia areata, ankylosing spondylitis, antiphospholipid syndrome, autoimmune Addison's disease, autoimmune hemolytic anemia, autoimmune hepatitis, autoimmune lymphoproliferative syndrome, autoimmunethrombocytopenic purpura, Behcet's disease, bullous pemphigoid, cardiomyopathy, celiac sprue-dermatitis herpetiformis, chronic fatigue immune dysfunction syndrome, chronic inflammatory demyelinating polyneuropathy, cicatricial pemphigoid, CREST syndrome, cold agglutinin disease, Crohn's disease, Degos' disease, dermatomyositis, dermatomyositis-juvenile, discoid lupus, essential mixed cryoglobulinemia, fibromyalgia-fibromyositis, Graves' disease, Guillain-Barré syndrome, Hashimoto's thyroiditis, idiopathic pulmonary fibrosis, idiopathic thrombocytopenia purpura, IgA nephropathy, insulin dependent diabetes, juvenile arthritis, lichen planus, lupus, Ménière's disease, mixed connective tissue disease, multiple sclerosis, myasthenia gravis, pemphigus, pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, Raynaud's phenomenon, Reiter's syndrome, rheumatic fever, rheumatoid arthritis, sarcoidosis, scleroderma, Sjögren's syndrome, stiff-man syndrome, Takayasu arteritis, temporal arteritis/giant cell arteritis, transplant rejection, ulcerative colitis, uveitis, vasculitis, vitiligo, and Wegener's granulomatosis.

16. The method of claim 13, wherein the pharmaceutical composition comprises the peptide of claim 3, and wherein the autoimmune disease or disorder is selected from bullous pemphigoid, idiopathic thrombocytopenia purpura, myasthenia gravis, pemphigus, pemphigus vulgaris, and transplant rejection.

17. A method of detecting FcRn, comprising:
labeling the peptide of claim 1 with a detectable label chosen from radioisotopes, enzymes having detectable products, fluorophores, chemiluminescent compounds, magnetic particles, microspheres, nanospheres, biotin, streptavidin, and digoxin.

18. A method of purifying FcRn, comprising:
(a) immobilizing the peptide of claim 1 to a solid support,
(b) contacting a solution containing FcRn with the immobilized peptide or conjugate on a solid support; and
(c) purifying FcRn by separating the solution from said solid support.


SEQUENCE LISTING (4 sequences)

SEQ ID NO: 1 - protein, 14 aa, Artificial Sequence
  Description of Artificial Sequence Synthetic peptide
RFXTGHFGXLYPCK

SEQ ID NO: 2 - protein, 13 aa, Artificial Sequence
  Description of Artificial Sequence Synthetic peptide
RFXTGHFGXLYPC

SEQ ID NO: 3 - protein, 12 aa, Artificial Sequence
  Description of Artificial Sequence Synthetic peptide
FXTGHFGXLYPC

SEQ ID NO: 4 - protein, 14 aa, Artificial Sequence
  Description of Artificial Sequence Synthetic peptide
RFXTGHFGXLYPCR